(19) United States
(12) Patent Application Publication
(10) Pub. No.: US 2004/0267317 A1
(43) Pub. Date: Dec. 30, 2004

(54) Methods for attaching tissue to bone

(76) Inventors: Laurence Higgins, Durham, NC (US); Izi Bruker, Wayland, MA (US); Jose Lizardi, Franklin, MA (US)

Correspondence Address: NUTTER MCCLENNEN & FISH LLP, WORLD TRADE CENTER WEST, 155 SEAPORT BOULEVARD, BOSTON, MA, 02210-2604, US

(21) Appl. No.: 10/607,322

(22) Filed: Jun. 26, 2003

(51) Int. Cl. A61B 17/04

(52) U.S. Cl. 606/232

(57) ABSTRACT

A method is provided for attaching tissue to bone. In general, the method includes the steps of providing a plurality of bone anchors, each having at least one channel formed therein for receiving suture, and threading a length of suture through at least one channel in at least one of the plurality of bone anchors. Each bone anchor can then be sequentially deployed through tissue and into bone as the free or terminal ends of the suture are preferably tensioned.

### FIELD OF THE INVENTION

The present invention relates to methods and devices for attaching tissue to bone.

### BACKGROUND OF THE INVENTION

Many surgical procedures require the attachment of soft tissue, e.g., ligament or tendon grafts or prostheses, to bone. This is typically accomplished by anchoring a suture in bone, for example with a screw, pin, or other bone anchoring device, and looping the suture around or stitching the suture through the soft tissue. Several different techniques are used for anchoring the tissue to the bone. In one attachment procedure, a receiving hole is first drilled into the bone, and then a bone anchor is inserted into the hole using an installation tool that is effective to lock the bone anchor within the bone hole. The free ends of the suture can then be passed through or around tissue and tied to secure the tissue to the bone. Alternatively, in some soft tissue reattachment procedures, the soft tissue can be moved into position over the bone, and a single hole may be drilled through both the tissue and the bone. A bone anchor can then be passed through the tissue and deployed into the bone. The free end of the suture is then tied to secure the tissue to the bone. In either procedure, the knotting process can be difficult and tedious, particularly during laparoscopic or endoscopic procedures, where the surgeon must remotely manipulate the suture using tools inserted through an access tube. As many as six knots may be required to secure one suture. Further, such procedures often require the use of several bone anchors to securely attach the tissue to bone and prevent the risk of tearing.

Devices have been developed which provide for the knotless application of a bone anchor. Such devices typically require a length of suture to be passed through tissue to form a loop with the free ends of the suture extending through the bone anchor. The anchor can then be deployed into bone, the suture tensioned to secure the tissue to the bone, and the anchor locked to prevent movement of the suture.

While knotless bone anchors are effective, reattachment procedures still require the placement of several bone anchors. Most current devices do not allow for several bone anchors to be implanted using a single length of suture, at least not without requiring the suture to be passed through tissue and threaded into a new anchor between each anchor application. Although there are mattress suture techniques known in this art that utilize anchors, the associated procedures may require repeated introduction and removal of an installation apparatus with the possible attendant disadvantages.

Accordingly, there remains a need for improved methods and devices for attaching tissue to bone.

### SUMMARY OF THE INVENTION

The present invention generally provides methods for attaching soft tissue to bone. In one embodiment, a plurality of bone anchors, each having at least one channel formed therein for receiving suture, are provided, and a length of suture is threaded through the at least one channel in at least one of the plurality of bone anchors. A first one of the plurality of bone anchors is deployed through tissue and into bone to cause the bone anchor to engage the suture and be embedded in the bone with a terminal length of suture extending out of the tissue and through the remaining, undeployed bone anchors. If each of the plurality of bone anchors is not pre-threaded with the suture, the terminal ends of the suture extending from the first bone anchor are then threaded through a second one of the remaining, undeployed bone anchors. The second bone anchor is then deployed through tissue into bone, at a location adjacent the position of the first bone anchor, to cause the bone anchor to engage the suture and be embedded in the bone with the terminal length of suture extending out of, the tissue and through the remaining, undeployed bone anchors. The steps of threading and deploying can be repeated as necessary. In an exemplary embodiment, each of the plurality of bone anchors is positioned in bone approximately 1 cm apart from one another.

In another embodiment, a method for attaching tissue to bone is provided using a bone anchor installation apparatus having a plurality of bone anchors disposed therein and a length of suture extending through each of the bone anchors. Each bone anchor is effective to engage the bone and the suture upon deployment into a bone hole. The installation apparatus can be actuated to apply a first bone anchor through tissue and into a first bone hole, and the apparatus can again be actuated to apply a second bone anchor through tissue and into a second bone hole adjacent to the first bone hole. The apparatus is preferably effective to tension the length of suture extending between the first and second bone anchors. The steps of actuating the apparatus to apply a plurality of bone anchors can be repeated, thereby securing tissue to the bone. In an exemplary embodiment, the installation apparatus is effective to move the bone anchor from an initial position, in which the suture is free to slide through at least one lumen formed in the anchor, to a final position, in which the bone anchor is deployed into a bone tunnel and the suture is engaged by the bone anchor. Preferably, each bone anchor includes an expandable body and an expander pin, and the step of actuating the apparatus is effective to drive the anchor into a bone tunnel, and to drive the expander pin into the expandable body to fix the suture with respect to the expander pin.

### BRIEF DESCRIPTION OF THE DRAWINGS

The invention will be more fully understood from the following detailed description taken in conjunction with the accompanying drawings, in which:

FIG. 1 is perspective view of one embodiment of a prior art suture anchor and delivery shaft for use with methods according to the present invention;

FIG. 2 is a perspective view of one embodiment of a prior art installation tool mated to the delivery shaft of FIG. 1 for use with methods according to the present invention;

FIG. 3 is an illustration of the step of inserting a delivery shaft having a suture anchor disposed thereon through tissue and into bone according to the present invention;

FIG. 4 is an illustration of the delivery shaft of FIG. 3 inserted through the tissue and into bone;

FIG. 5 is an illustration of an installation apparatus mated to the delivery shaft shown in FIG. 4;

FIG. 6 is an illustration of the bone anchor shown in FIG. 4 engaged within the bone;

FIG. 7 is an illustration of the step of threading the terminal ends of suture extending from the first bone anchor through a second bone anchor according to another embodiment of the present invention;

FIG. 8 is an illustration of two bone anchors deployed into bone according to yet another embodiment of the present invention; and

FIG. 9 is an illustration of tissue attached to bone using several bone anchors configured to provide optimal tissue-to-bone contact, according to another embodiment of the present invention.

### DETAILED DESCRIPTION OF THE INVENTION

In general, the present invention provides improved methods for attaching tissue to bone using several bone anchors that can be deployed into bone in a manner that is convenient and economical for surgeons. In particular, multiple bone anchors can be deployed into bone sequentially without the need for an additional step of suturing the tissue following deployment of each bone anchor. Moreover, the method can be achieved without removing the installation apparatus from the surgical site. Accordingly, the methods of the invention are particularly advantageous in that the procedure is relatively easy and efficient. Moreover, the procedure allows the contact area, e.g., the footprint, between the tissue and the bone to be optimized, thereby improving surgical results by reducing the risk of re-tearing the repaired tissue.

In order to understand the methods of the present invention, it is useful to first describe exemplary embodiments of bone anchors and installation tools that can be used to perform the methods of the invention. A person skilled in the art will appreciate that the methods of the present invention can be achieved using a variety of bone anchors and bone anchor installation tools, and that the bone anchors and other tools described herein are merely shown for illustration purposes, and are not intended to limit the scope of the invention. By way of non-limiting example, suitable bone anchors and installation tools are disclosed in the following patent and patent applications; each of which is incorporated herein by reference in its entirety: U.S. Pat. No. 6,319,252 of McDevitt et al., entitled “System and Method for Attaching Soft Tissue to Bone;” U.S. patent application Ser. No. 09/360,474, filed on Nov. 20, 2001, entitled “System and Method for Attaching Soft Tissue to Bone,” and U.S. patent application Ser. No. 09/510,770, filed on Feb. 23, 2000, entitled “System and Method for Attaching Soft Tissue to Bone.”

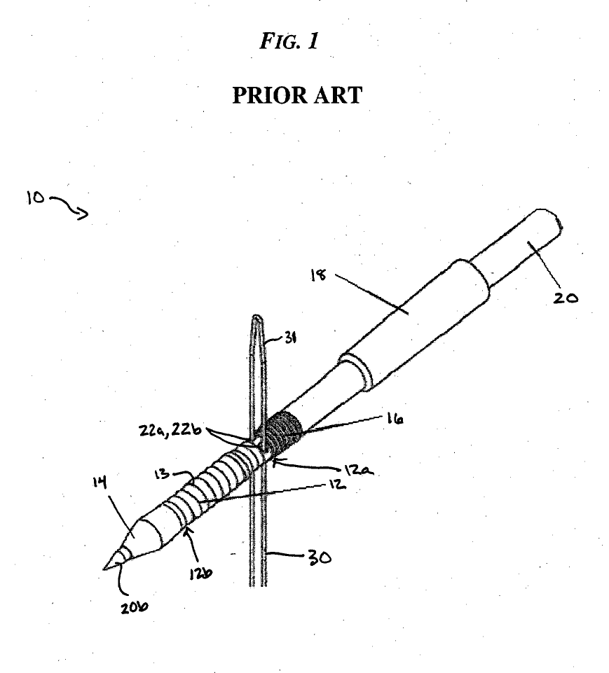

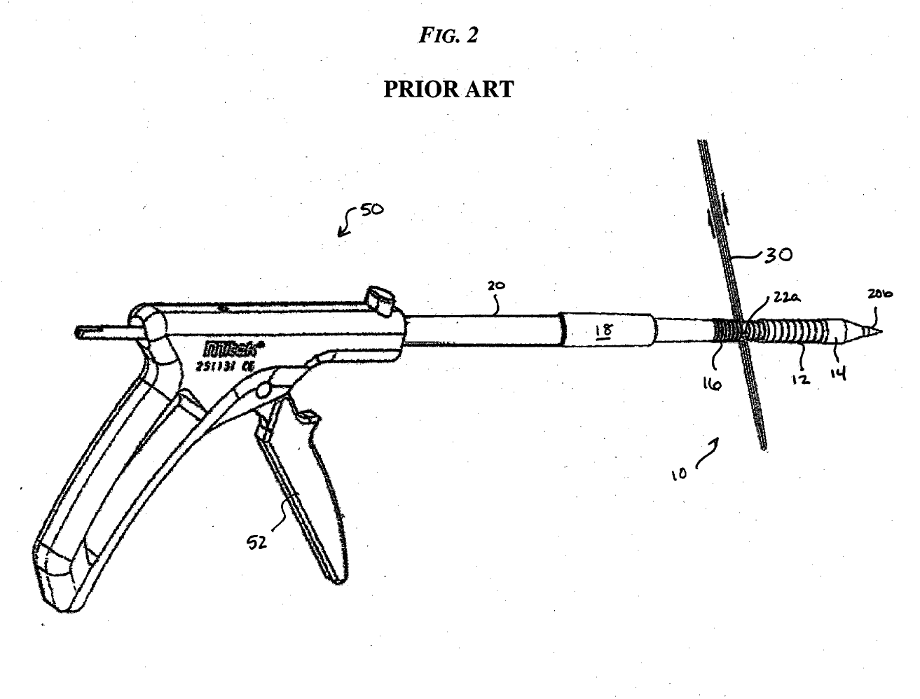

FIGS. 1 and 2 illustrate an exemplary embodiment of a bone anchor 10, a delivery shaft 20, and an installation tool 50 that can be used to perform the methods of the present invention. As shown in FIG. 1, the bone anchor 10 generally includes an expandable sleeve 12 and an expander pin 16 that is adapted to be disposed within and to expand the expandable sleeve 12. The expandable sleeve 12, which is attached to a delivery shaft 20 that is adapted for use with the installation tool 50, can have a variety of configurations, but it preferably has a generally elongate cylindrical shape with a proximal end 12a, a distal end 12b, and an inner lumen (not shown) extending therebetween. One or more surface features can be formed on at least a portion of the sleeve 12 to facilitate engagement between the expandable sleeve 12 and a bone tunnel. While the surface features can have a variety of configurations, FIG. 1 illustrates a series of ridges 13 formed around the expandable sleeve 12.

The expandable sleeve 12 can also include one or more suture channels 22a, 22b extending therethrough to receive a length of suture. In an exemplary embodiment, as shown, the expandable sleeve 12 includes two transverse suture channels 22a, 22b extending through the proximal end 12a thereof to allow a suture loop 31 to be formed. The location of the suture channels 22a, 22b allows a suture 30 to be retained by interference between the expandable sleeve 12 and the expander pin 16 when the bone anchor 10 is fully deployed. A person skilled in the art will appreciate that a variety of techniques can be used to attach one or more lengths of suture 30 to the expandable sleeve 12, and that a variety of techniques can be used to engage or lock the suture with respect to the device 10 when the device 10 is fully implanted in bone.

In order to insert the expandable sleeve 12 into bone, a bone tunnel can be drilled into bone to receive the sleeve 12, and/or the expandable sleeve 12 can be self-tapping or self-piercing. In an exemplary embodiment, the distal end 12b of the expandable sleeve 12 includes a frusto-conical distal tip 14 to facilitate insertion of the expandable sleeve 12 into bone. The frusto-conical tip 14 allows a sharpened distal tip 20b of the delivery shaft 20 (discussed in more detail below) to be inserted therethrough to facilitate insertion of the anchor 10 into bone. The distal tip 14 can be fixedly attached to the expandable sleeve 12, but preferably it is threadably mated to the distal end 12b of the expandable sleeve 12. A primary advantage of forming the distal tip 14 out of a separate component from the expandable sleeve 12 is that each component can be optimized for its own function. More particularly, inasmuch as the distal tip 14 is intended to help open a passageway in bone to receive the expandable sleeve 12, the distal tip 14 can be formed out of a relatively rigid material. At the same time, however, the expandable sleeve 12 can be formed out of a relatively soft, expandable material to allow the expandable sleeve 12 to expand radially outward during deployment so as to fix the expandable sleeve 12 in bone.

As indicated above, the bone anchor 10 also includes an expander pin 16 that is adapted to expand the expandable sleeve 12. The expander pin 16 can have a variety of shapes of sizes, but preferably the expander pin 16 has a generally cylindrical shape and it is sized so that when the expander pin 16 is driven longitudinally into the expandable sleeve 12, the expander pin 16 will force the sidewalls of the expandable sleeve 12 to expand radially outwardly against adjacent bone, thereby securing the bone anchor 10 in the bone tunnel. The expander pin 16 can be fixedly attached to, integrally formed with, or removably mated to the expandable sleeve 12. In an exemplary embodiment, however, the expander pin 16 is formed as a separate component from the expandable sleeve 12. While not shown, a distal end of the expander pin 16 is preferably tapered to facilitate insertion of the expander pin 16 into the expandable body. As stated above with respect to the distal tip 14, forming the expander pin 16 as a separate component from the expandable sleeve 12 likewise allows the expander pin 16 to be formed from a material that is more rigid than the material used to form the expandable sleeve 12.

FIG. 1 further illustrates a delivery shaft 20 that is effective to couple to the installation tool 50, and that can be used to insert the bone anchor 10 into bone. The delivery shaft 20 can have a variety of configurations, but in an exemplary embodiment it is generally elongate and cylindrical in shape. The shaft 20 may include a sharpened distal tip 20b that can be inserted through the expander sleeve 12 and expander pin 16, and that is used to tap the device 10 into a bone tunnel. A pusher member 18 can be slidably disposed on the shaft 20, proximal to the bone anchor 10, to push the expander pin 16 into the expandable sleeve 12. The pusher member 18 can be moved along the shaft 20 using installation tool 50, as will be discussed below. The delivery shaft 20 also preferably includes threads (not shown) formed around a distal portion thereof that allows the shaft 20 to mate with corresponding threads (not shown) formed within a portion of the expandable sleeve 12. The threads are effective to maintain the position of the expandable sleeve 12 with respect to the shaft 20 to allow the entire assembly to be inserted into bone, and to allow the expander pin 16 to be pushed into the expandable sleeve 12.

Referring now to FIG. 2, an exemplary embodiment of an installation tool 50 is shown mated to the delivery shaft 20. In general, the installation tool 50 includes a trigger member 52 that, upon actuation, is effective to advance the pusher member 18 distally along the delivery shaft 20, thereby forcing the expander pin 16 into the expandable sleeve 12. Specific details regarding the installation tool 50 are set forth in U.S. patent application Ser. No. 09/510,770, entitled “System and Method for Attaching Soft Tissue to Bone,” filed on Feb. 23, 2000, which is incorporated herein in its entirety. A person skilled in the art will appreciate that a variety of other techniques can be used to actuate the installation tool 50 and to advance the pusher member 18 to deploy the expander pin 16 into the expandable sleeve 12.

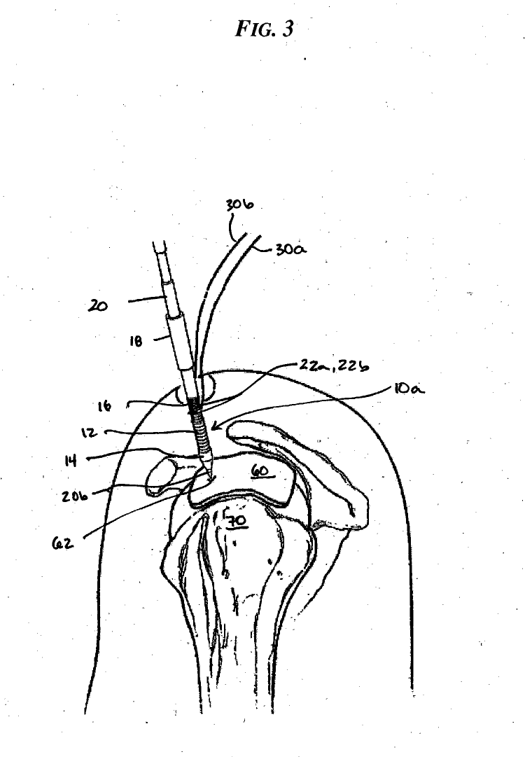

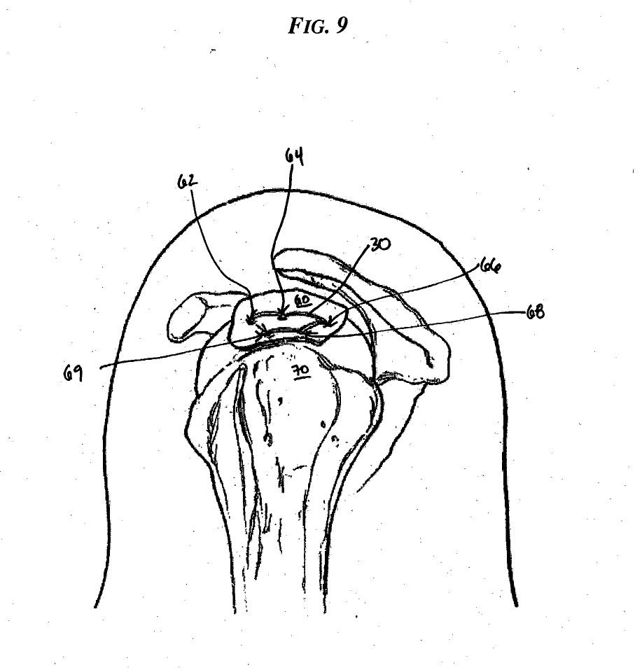

Referring now to FIGS. 3-9, an exemplary method of attaching tissue to bone is shown. While the method is described in connection with bone anchor 10, delivery shaft 20, and installation tool 50, virtually any bone anchor and delivery shaft and/or installation tool (if necessary) can be used to perform the method. Moreover, while the method is applicable to connecting detached tissue in other contexts, the method described herein, in an exemplary embodiment, is used in the context of an arthroscopic shoulder repair, and more specifically, for attaching a detached or torn supraspinatus tendon to the humeral head (as in a rotator cuff repair).

As shown in FIG. 3, the procedure generally requires a first bone anchor 10a to be placed on the delivery shaft 20 with a length of suture 30 threaded through the suture channels 22a, 22b in the expandable sleeve 12 such that two free ends 30a, 30b of the suture 30 extend therefrom. The suture 30 can be mated to the anchor using a variety of other techniques known in the art. The delivery shaft 20 with the anchor 10 loaded thereon is then preferably introduced into the surgical site using a delivery cannula. The distal tip 20b of the shaft 20, depending on the configuration, can then be driven into tissue 60 and used to position the tissue 60 at the desired location against the bone 70. Alternatively, tissue 60 can be gripped by another instrument (e.g., forceps or the like) and moved into position against the bone 70.

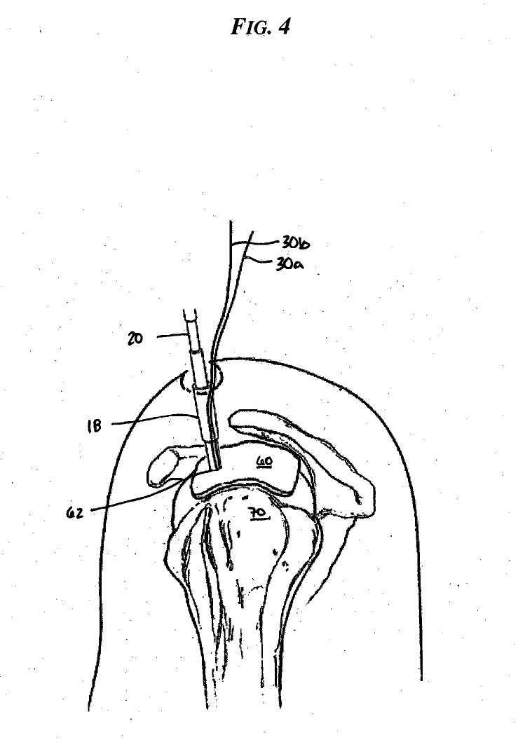

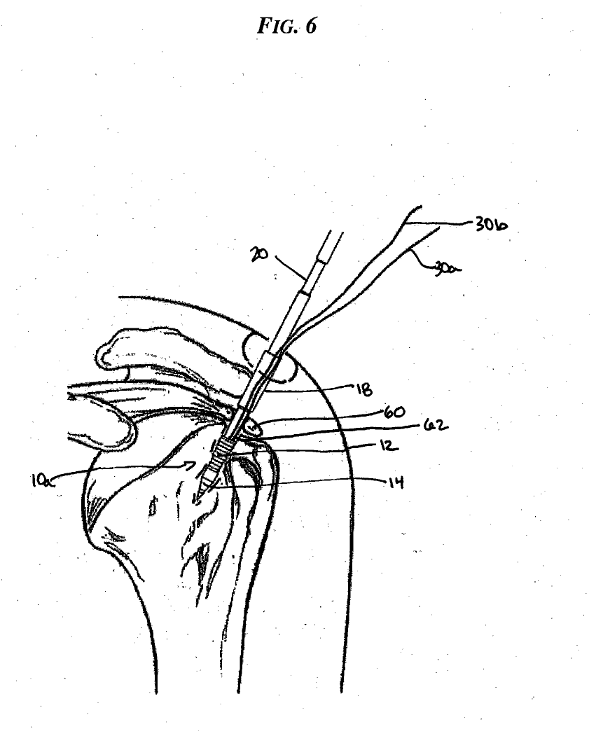

With the tissue 60 in position against the bone 70, the shaft 20 can be forced distally through the tissue 60 and into the bone 70, as shown in FIG. 4, at the desired tissue attachment site (hereinafter referred to as the first tissue attachment site 62). It will be appreciated that, as this occurs, expandable sleeve 12 will be carried into the bone 70 due to the threaded engagement established between the expandable sleeve 12 and the shaft 20. In fact, the tapered distal end 20b of the shaft 20 and the distal tip 14 of the expandable sleeve 12 will cooperate with one another so as to force an opening in the tissue 60 and the bone 70, preferably without any need for pre-drilling. The shaft 20 is preferably driven into bone 70 until the proximal end 12a of the expandable sleeve 12 is approximately flush with the outer surface of bone 70 (FIG. 6). If desired, markings (not shown) may be placed on the outer surface of the fastener's expander pin 16 so that, once the thickness of the tissue 60 is known, proper depth penetration can be achieved.

A person skilled in the art will appreciate that the method of inserting the anchor into bone will depend on the configuration of the particular anchor being implanted. By way of non-limiting example, where the anchor is not tissue-penetrating or self-tapping, a bone tunnel can be predrilled into bone for receiving the anchor.

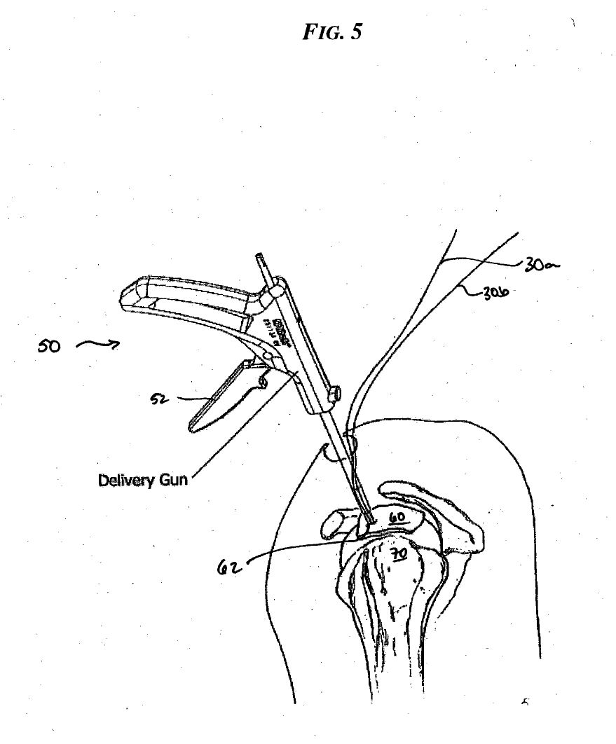

Once the bone anchor is inserted into the bone tunnel, the delivery shaft 20 is attached to the installation tool 50, as shown in FIG. 5. The trigger 52 can then be actuated to drive the pusher 18 distally along the shaft 20, thereby driving the expander pin 16 into the expandable sleeve 12. As a result, the sidewalls of the expandable sleeve 12 will expand radially outward into bone 70( to secure the anchor 10 to the bone 70 as shown in FIG. 6. In this configuration, the suture 30 is secured between the expander pin 16 and the expandable sleeve 12. The shaft 20 and installation tool 50 can then be removed. In a procedure where only one anchor 10a is used to attach the tissue 60 to the bone 70, the free ends 30a, 30b of the suture would, at this point, be tied to secured to the tissue 60 to the bone 70. However, the present invention provides an improved method for attaching tissue to bone in which several anchors are sequentially deployed.

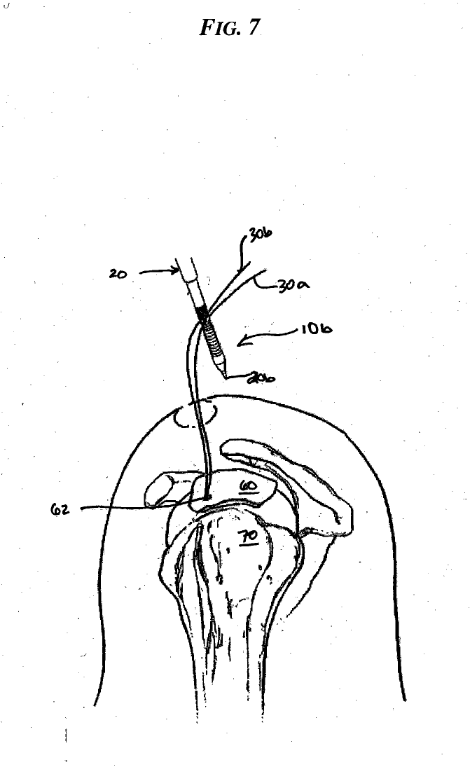

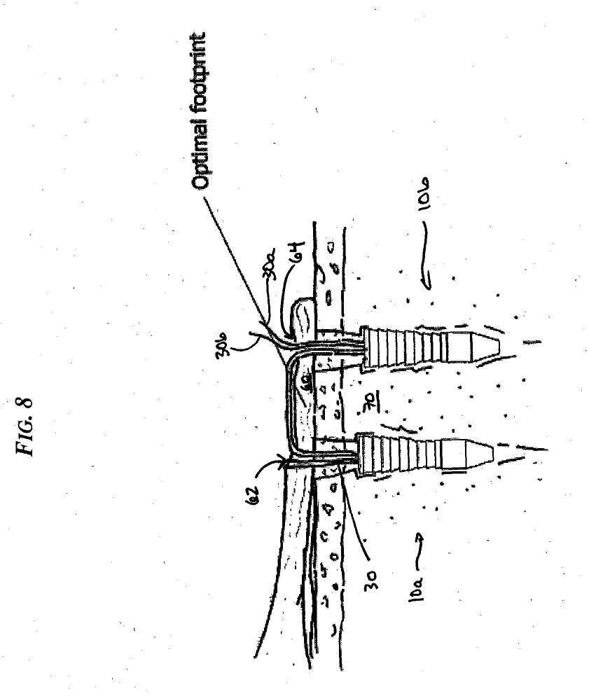

Accordingly, a second anchor 10b can be attached to the delivery shaft 20, as shown in FIG. 7. The free or terminal ends 30a, 30b of the suture 30, which extend from the first tissue attachment site 62, are threaded through the second anchor 10b, and the distal tip 20b of the delivery shaft 20 is then inserted through tissue 60 at a second tissue attachment site 64. In an exemplary embodiment, as shown in FIG. 8, the second, tissue attachment site 64 is positioned about 1 cm apart from the first tissue attachment site 62. Once properly positioned, the shaft 20 is forced distally through the tissue 60 and into the bone 70, and the installation tool 50 is then used to deploy the expander pin 16 into the expandable sleeve 12, thereby locking the second anchor member 10b in bone 70 at the second tissue attachment site 64. The free or terminal ends 30a, 30b of the suture 30 are preferably tensioned as the second anchor 10b is inserted into bone 70 and/or as the installation tool 50 is actuated. As shown, a tissue-to-bone contact area, e.g., a “footprint,” is formed between the two anchors 10a, 10b.

The aforementioned procedures can be repeated using several anchors, each connected by the same suture 30. Once all of the desired suture anchors are implanted, the free or terminal ends 30a, 30b of the suture 30 can be tied and/or trimmed.

In another embodiment of the present invention, one or more anchors can be pre-threaded with a length of suture 30 that extends through all of the anchors. More particularly, the delivery shaft 20 and/or the installation tool 50 can be configured to retain several anchors 10 thereon such that the free ends 30a, 30b of the suture 30 can be threaded through all of the anchors 10. By way of non-limiting example, the delivery shaft can be configured preloaded with several anchors such that the suture extends through the lumen in each anchor, and the delivery shaft or the installation tool can be adapted to advance one anchor at a time along the shaft for deployment. Each anchor 10 can then be individually deployed into bone without the need to thread the terminal ends 30a, 30b of the suture 30 into the next bone anchor 10 prior to deployment of the bone anchor 10. The installation tool 50 and/or delivery shaft 20 can also optionally be configured to tension the terminal ends 30a, 30b of the suture 30 during deployment of each anchor 10. Accordingly, the method can be performed relatively efficiently, and without the need to perform any additional threading steps.

One of ordinary skill in the art will appreciate further features and advantages of the invention based on the above-described embodiments. Accordingly, the invention is not to be limited by what has been particularly shown and described, except as indicated by the appended claims. All publications and references cited herein are expressly incorporated herein by reference in their entirety.

What is claimed is:

1. A method for attaching soft tissue to bone, comprising the steps of:
providing a plurality of bone anchors, each having a suture-receiving member formed therein or thereon;
attaching a length of suture to the suture-receiving member formed on each of the plurality of bone anchors;
deploying a first one of the plurality of bone anchors into bone to cause the bone anchor to engage the suture and be embedded in the bone with a terminal length of suture extending out of the bone and being attached to the remaining plurality of bone anchors;
deploying a second one of the plurality of bone anchors into bone, at a location adjacent the position of the first bone anchor, to cause the bone anchor to engage the suture and be embedded in the bone with the terminal length of suture extending out of the bone and being attached to the remaining plurality of bone anchors; and
repeating the step of deploying as necessary.

2. The method of claim 1, wherein each bone anchor is deployed through tissue prior to deploying the bone anchor into bone.

3. The method of claim 1, wherein the suture-receiving member comprises at least one channel formed in each of the plurality of bone anchors, and wherein the step of attaching the suture comprises threading the suture through at least one channel in each of the plurality of bone anchors.

4. The method of claim 3, wherein each bone anchor comprises:
an expandable body defining a bore formed therein; and
an expander pin adapted to be received within the bore in the expandable body, the at least one channel extending through the expander pin.

5. The method of claim 4, wherein the step of deploying a bone anchor through tissue and into bone to cause the bone anchor to engage the suture and be embedded in bone comprises the step of driving the expander pin into the expandable body to fix the suture with respect to the expander pin.

6. The method of claim 5, wherein the suture is held by interference between the expander pin and the expandable body.

7. The method of claim 1, wherein each of the plurality of bone anchors are positioned in bone approximately 1 cm apart from one another.

8. A method for attaching soft tissue to bone; comprising the steps of:
providing a plurality of bone anchors, each having at least one channel formed therein for receiving suture;
threading a length of suture through at least one channel formed in a first one of the plurality of bone anchors;
deploying the first one of the plurality of bone anchors through tissue and into bone to cause the bone anchor to engage the suture and be embedded in the bone with a terminal length of suture extending out of the tissue and through the remaining plurality of bone anchors;
threading a length of suture through at least one channel formed in a second one of the plurality of bone anchors;
deploying a second one of the plurality of bone anchors through tissue into bone, at a location adjacent the position of the first bone anchor, to cause the bone anchor to engage the suture and be embedded in the bone with the terminal length of suture extending out of the tissue and through the remaining plurality of bone anchors; and
repeating the step of threading and deploying as necessary.

9. The method of claim 8, wherein each bone anchor comprises:
an expandable body defining a bore formed therein; and
an expander pin adapted to be received within the bore in the expandable body, the at least one channel extending through the expander pin.

10. The method of claim 9, wherein the step of deploying a bone anchor through tissue and into bone to cause the bone anchor to engage the suture and be embedded in bone comprises the step of driving the expander pin into the expandable body to fix the suture with respect to the expander pin.

11. The method of claim 10, wherein the suture is held by interference between the expander pin and the expandable body.

12. The method of claim 9, wherein each of the plurality of bone anchors are positioned in bone approximately 1 cm apart from one another.

13. The method of claim 9, wherein the suture is tensioned during the step of deploying the a bone anchor into bone.

14. A method for attaching tissue to bone, comprising the steps of:
providing a bone anchor installation apparatus having a plurality of bone anchors disposed therein and a length of suture extending through each of the bone anchors, the bone anchors being effective to engage the bone and the suture upon deployment into a bone hole;
actuating the installation apparatus to apply a first bone anchor through tissue and into a first bone hole;
actuating the apparatus to apply a second bone anchor through tissue and into a second bone hole adjacent the first bone hole, the apparatus being effective to tension the length of suture extending between the first and second bone anchors;
repeating the step of actuating the apparatus to apply a plurality of bone anchors, thereby securing tissue to the bone.

15. The method of claim 14, wherein the installation apparatus is effective to move the bone anchor from an initial position, in which the suture is free to slide through at least one lumen formed in the anchor, to a final position, in which the bone anchor is deployed into a bone tunnel and the suture is engaged by the bone anchor.

16. The method of claim 14, wherein each bone anchor comprises:
an expandable body defining a bore formed therein; and
an expander pin adapted to be received within the bore in the expandable body, the at least one channel extending through the expander pin.

17. The method of claim 16, wherein the step of actuating the apparatus is effective to drive the anchor into a bone tunnel, and to drive the expander pin into the expandable body to fix the suture with respect,to the expander pin.

18. The method of claim 14, wherein each bone anchor is positioned in bone approximately 1 cm apart from one another.
